(19) United States
(12) Patent Application Publication
(10) Pub. No.: US 2002/0026093 A1
(43) Pub. Date: Feb. 28, 2002

(54) Endscope system

(75) Inventor: Masayuki Ooyatsu, Tochigi-ken (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: KABUSHIKI KAISHA TOSHIBA, TOKYO (JP)

(21) Appl. No.: 09/934,530

(22) Filed: Aug. 23, 2001

(30) Foreign Application Priority Data
Aug. 23, 2000 (JP) P2000-251852

(51) Int. Cl. A61B 1/045

(52) U.S. Cl. 600/118; 600/167

(57) ABSTRACT

An endoscope system including a solid-state image pickup device having a photosensitive area and configured to produce an electronic image; an optical system having a focal length and configured to transmit an optical image of an object incident onto the photosensitive area; a superimposing device configured to superimpose on the electronic image produced by the solid-state image pickup device scale information according to the focal length of the optical system; and a display configured to display the electronic image and the superimposed scale information. The focal length of the optical system is variable by means of a lever operated wire connected to a lens of the optical system and disposed in an endoscope inserter. Thus, an operator can easily measure the length of the object based on the scale information superimposed on the displayed electronic image.

### BACKGROUND OF THE INVENTION

1. Field of the Invention

The present invention generally relates to an electronic endoscope system, and more specifically, to an electronic endoscope system capable of measuring a size of an object. This application is based upon and claims the benefit of priority from Japanese Patent Application No. 2000-251852, filed on Aug. 23, 2000, the entire contents of which are incorporated herein by reference.

2. Discussion of Background

An endoscope system has been widely utilized for medical diagnosis or treatment. In diagnosis or treatment, an operator sometimes measures a size of an object inside a body with the endoscope.

With a conventional endoscope, the operator has to use forceps for inserting a graduated band into a body cavity, put it near the object and compare a graduation on the band with the object. Both the graduation on the band and the object are displayed on the same monitor so that the operator can measure the size of the object.

Such a graduated band is described in Japanese KOKAI (Disclosure) patent application No. 63-277028. However, it is often difficult to put the graduated band near the object due to the movement of the body cavity, which may cause a long measurement time. Additionally, the graduated band may be an obstacle to observe the object.

Moreover, there is an endoscope system for magnifying the object by changing a focal length of an optical system disposed in an endoscope unit. The optical system like this is designed such that a depth of field is small when the object is magnified.

The operator has to move the endoscope unit such that the object is located at the position within the depth of field. In such a case, it is difficult to put the graduated band near the object because a field of view is small. Further the size of the object cannot be measured correctly depending upon the experience of the operator.

A technique that solves the above-described problems is disclosed in Japanese KOKAI (Disclosure) patent application No. 59-69721, which discloses an endoscope having a laser system for measuring a distance between an end of the endoscope unit and the object and superimposing a scale on an image of the object according to the distance. However this endoscope system is complicated and expensive due to the laser system. Further, it cannot measure the desired distance if the object to be observed is not actually irradiated by a laser beam.

### SUMMARY OF THE INVENTION

To solve these problems, an object of the present invention is to provide an endoscope system with which the operator can measure a size of an object easily.

In order to accomplish this object, the present invention provides an endoscope system including a solid-state image pickup device having a photosensitive area and configured to produce an electronic image; an optical system having a focal length and configured to transmit an optical image of an object incident onto the photosensitive area; a superimposing device configured to superimpose on the electronic image produced by the solid-state image pickup device scale information according to the focal length of the optical system; and a display configured to display the electronic image and the superimposed scale information.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a general diagram showing an embodiment of the endoscope system according to the present invention;

FIG. 2 is a longitudinal cross-sectional view showing an end of the endoscope unit of the embodiment shown in FIG. 1;

FIG. 3 is a block diagram showing a system configuration of the embodiment of FIG. 1;

FIG. 4 is a flowchart of the procedure of operating the endoscope system of FIG. 1;

FIG. 5 is an illustration of an image of an object displayed on a monitor in a normal mode as a normal picture;

FIG. 6 is an illustration of an image of an object displayed on a monitor in an enlargement mode to provide a zoom-in picture;

FIG. 7 is an illustration of an image of the object displayed with a superimposed scale;

FIG. 8 is an illustration of a second example of displayed scale information;

FIG. 9 is an illustration of a third example of displayed scale information;

FIG. 10 is a graph showing a relation between a distance from the center on a monitor and an actual length of the object;

FIG. 11 is an illustration of another example of displayed scale information;

FIG. 12 is an illustration of another example of displayed scale information;

FIG. 13 is an illustration of an object image on which scale information is superimposed;

FIG. 14 is an illustration of an object image and superimposed scale information resulting upon moving of the scale information relative to the object image.

FIG. 15 is an illustration showing a menu for setting up of the system regarding storing the scale information; and

FIG. 16 is a longitudinal cross-sectional view showing an operating handle of an endoscope unit.

### DETAILED DESCRIPTION OF THE INVENTION

Referring now to the drawings, wherein like reference numerals designate identical or corresponding parts through the several views, an embodiment according to the present invention will now be described.

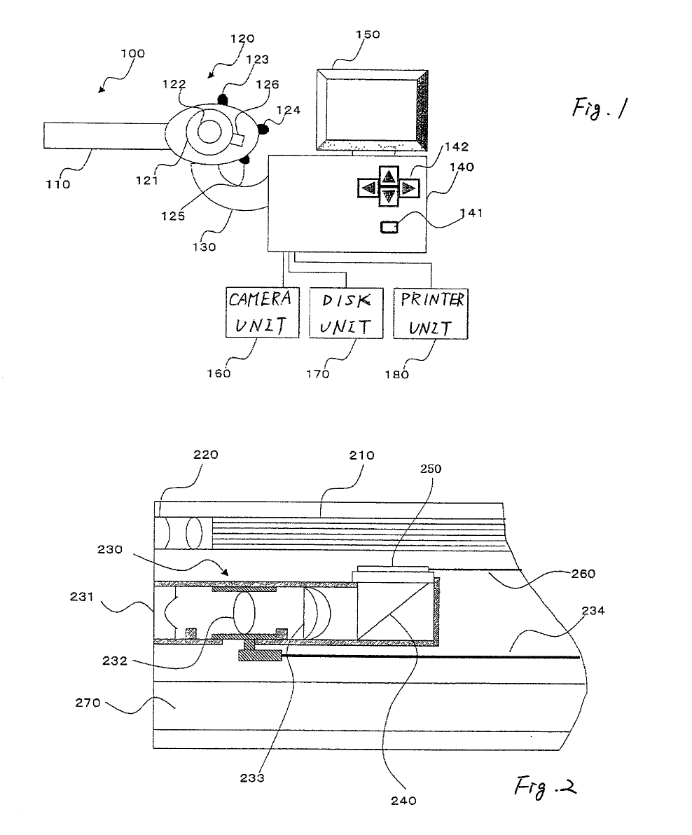

FIG. 1 is a general view showing an embodiment of the endoscope system according to the present invention, including an endoscope unit 100 to be inserted into a body cavity being examined to pick up an image for medical diagnosis, a main unit 140 connected to the endoscope unit 100 through a universal cord 130, and a monitor 150 connected to the main unit 140 to display an electronic image of the body cavity. The universal cord 130 transmits image signals obtained by the endoscope part 100 to the main unit 140 and light from a light source unit to the endoscope unit 100. The endoscope unit has an operating handle 120 and an inserter 110. The operating handle 120 includes a first knob 121 to bend an end of the inserter 110 up or down, and a second knob 122 for curving the end to the right or the left, respectively. An operator rotates both knobs 121, 122 to search an object or region of interest. In the operating handle a lock lever is provided to lock movement of the both knobs.

The main unit 140 may also be connected to a variety of recording devices, such as a camera unit 160, a disk unit 170 and a printer unit 180 each for recording the electronic image. The camera unit 160 records the image onto a film, the disk unit 170 stores the image into a disk such as hard disk, a CD-R, a DVD or a magneto-optical disk. Moreover the printer unit 180 records the image by printing the image onto a paper receptor.

The operating handle 120 has switches 123, 124 and 125. The switch 123 is used when a moving image is changed to a still image (freezing), the switch 124 is used when the still image is stored into at least one of the camera unit 160 and the disk unit 170. The switch 125 is used when the still image is printed by the printer unit 180.

With a switch (not shown) disposed at the main unit 140 the operator can select whether the still image is stored into the camera unit 160 or the disk unit 170 when the switch 124 is operated.

During operation, the operator freezes the image using the switch 123 and then pushes the switch 124 or 125 to record the still image in by means of the camera unit 160, the disk unit 170 or the printer unit 180. Alternatively, the switch 124/125 can be set to initiate freezing and recording images without pressing the switch 123. Furthermore the operator can select whether the still image is changed to the moving image automatically or not after storing. Such selections are made with switches (not shown) disposed on the main unit 140.

The main unit 140 further includes a switch 141 for triggering the timing when scale information indicating a graduation is displayed with the image on the monitor 150, and a directional switch 142 for moving the position of the scale information on the monitor 150. The scale information is described below. The operating part 120 further includes a lever 126 for changing the optical magnification of the image. The operator rotates the lever 126 to select for image display a zoom-in or a regular normal picture. In this embodiment the magnification is selectable from two levels.

The magnification of the image is described herein below with reference to FIG. 2. FIG. 2 is a longitudinal cross-sectional view showing an end of the inserter 110 of the endoscope unit 100. A light guide 210 is optically connected to the light source unit and transmits a light to the end of the inserter 110. The light is emitted through the lens 220 disposed at the end of the inserter 110.

An optical image of the body cavity illuminated by the light goes through an optical system 230 and a prism 240 and arrives at a photosensitive area disposed on a CCD (Charge Coupled Device) 250. The optical image is transformed to an electronic image signal by the CCD 250, and fed to the main unit 140 through an electric cable 260 electrically connected to the CCD 250. The inserter 110 has a channel 270 through which a surgical instrument, such as forceps, is disposed.

The optical system 230 includes a first lens 231, a second lens 232 and a third lens 233. The first lens 230 is fixed at an end of the inserter 110 and the third lens 233 is located between the first lens 231 and the prism 240. The second lens 233 is disposed between the first lens 231 and the third lens 233 and is mechanically connected to an end of a wire 234. The other end of the wire 234 is connected to the lever 126. The lens 232 is movable along the longitudinal direction according to the rotation of the lever 126, which changes a focal length of the optical system 230.

In this embodiment, the optical system is varifocal and designed such that a depth of field becomes smaller to the optical system and the focal length becomes larger when the second lens 232 moves to the first lens 231. In this case, the image is displayed as a zoom-in picture.

On the other hand, the depth of field becomes far from the optical system and the focal length becomes smaller when the second lens 232 moves to the third lens 233. In this case, the image is displayed as a normal picture.

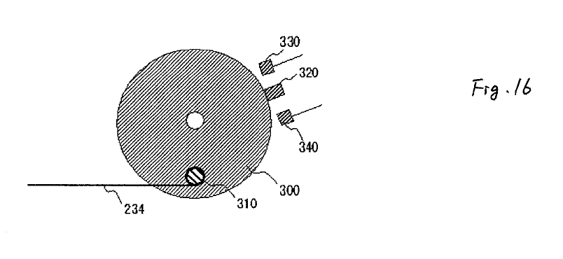

A sensor for detecting a position of the lever 126 that is used to change the focal length of the optical system is described with reference to FIG. 16, which is a longitudinal cross-sectional view showing an operating handle of an endoscope unit. A disk 300 is disposed in the operating handle 120 and fixed to the lever 126. The disk 300 has a projection 310 projecting vertically on a surface of the disk 300. The projection 310 is wound with the wire 234 connected to the second lens 232. Moreover the disk 300 has a projection 320 that protrudes horizontally on a surface of the disk 300. Switches 330 and 340 are located on opposite sides of the projection 320 in a movable direction of the disk 300, respectively. When the projection 320 touches the switch 330, the image is displayed as a normal picture on the monitor and when the projection 320 touches the switch 340 a zoom-in picture is displayed. The signal from each switch 330, 340 is fed to the main unit 140, respectively.

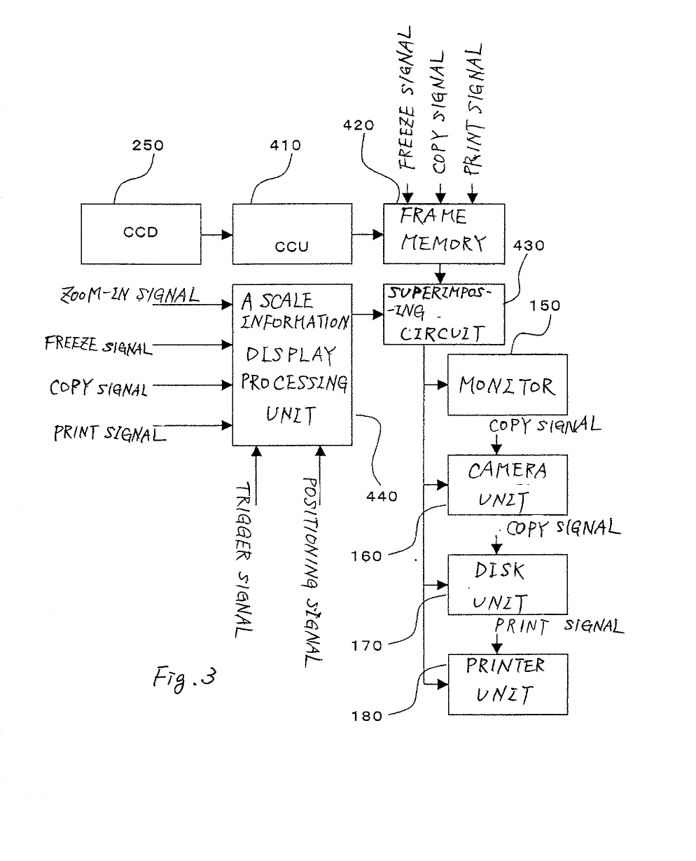

Next, the operation of this embodiment will be described with reference to FIG. 3, which is a block diagram showing an embodiment of the system configuration. The optical image of the body cavity illuminated by the light passes through the optical system 230 and the prism 240 and arrives at the photosensitive area disposed on the CCD 250. The electronic image signal converted from the optical image by the CCD 250 is fed to a CCU 410 (camera control unit) that is disposed in the main unit 140. The CCU 410 controls the CCD 250, for example a read-out timing of the CCD 250, and feeds the electronic image to a frame memory 420 that is also disposed in the main unit 140. The electronic image signals read out from the frame memory 420 pass through a superimposing circuit 430 that superimposes scale information on the electronic image which is then displayed as a moving image on the monitor 150.

A scale information display processing unit 440, disposed in the main unit 140, includes a memory in which various types of scale information is stored corresponding to each focal length of the optical system 110, and is used for determining the timing when the scale information is displayed according to some operations of the operator. The scale information stored in the main unit 140 is fed to the superimposing circuit 430 according to operations described below and then displayed on the monitor 150. The scale information is determined according to the focal length of the optical system 110.

During operation, the operator rotates the lever 126 in order to change the focal length such that the image is displayed as zoom-in picture and moves the endoscope unit 100 back and forth to position an observation area of interest within the depth of field. If necessary, the operator may push one of the switches 123, 124 and 125 in order to freeze the image, store the image, or print the image, respectively.

In this embodiment, there are four types of operations that generate the timing when the scale information is displayed. These operations are described below.

(1) When the image is displayed as a zoom-in picture:

The operator rotates the lever 126 then the disk 300 shown in FIG. 16 is rotated to the same direction as the lever 126. The projection 310 is also rotated with the disk 300 and pulls or pushes the second lens 232 by means of the wire 234 to change the focal length. Additionally, the projection 320 is also rotated and touches the switch 330 or the switch 340. One of the switches is turned ON when touched by the projection 320 and an output of the switch is fed to the scale information display processing unit 440.

For example, if the operator rotates the lever 126 clockwise, the second lens 232 is pushed toward the first lens 231 and the focal length is increased, which means the image is displayed as large picture. The projection 320 is also rotated and turns the switch 340 ON whose output is fed to the scale information display processing unit 440. The scale information display processing unit 440 outputs the scale information to the superimposing circuit 430. Then, the superimposing circuit 430 superimposes the scale information on the image read-out from the frame memory 420 and the superimposed image is displayed on the monitor 150. The mechanism and manner to change and detect the focal length of the optical system are not limited to this embodiment, for example, the switch 330 may be omitted.

(2) When the switch only for triggering the timing is turned ON:

When the operator turns the switch 141 ON, the scale information display processing unit 440 receives a signal from the switch 141 and the output of the scale information display processing unit 440 is fed to the superimposing circuit 430. Then, the superimposing circuit 430 superimposes the scale information on the image read-out from the frame memory 420 and the superimposed image is displayed on the monitor 150.

(3) In the case the timing is made when the switch for freezing the image turned ON:

When the operator turns the switch 123 ON, the frame memory 420 receives an image freeze signal and the moving image is changed to a still image. The scale information display processing unit 440 also receives the freeze signal and the output of the scale information display processing unit 440 is fed to the superimposing circuit 430 so that the superimposing circuit 430 then superimposes the scale information and the still image. In addition, a delay time may be set in the scale information display processing unit 440 such that the scale information is displayed, for example, a second after the switch 123 is turned ON. In this case the operator can confirm the still image displayed on the monitor is adequate before the scale is displayed on the monitor.

(4) When the switch for storing the image is turned ON:

When the operator turns the switch 124 or 125 ON, the scale information display processing unit 440 receives the storing signal. The output of the scale information display processing unit 440 is fed to the superimposing circuit 430 and the superimposing circuit 430 then superimposes the scale information on the still image. The frame memory 420 also receives a storing signal and the moving image is changed to the still image. The camera unit 160, a disk unit 170 or a printer unit 180 also receives the storing signal and the image with the scale information is recorded.

In addition, a delay time may be set in the scale information display processing unit 440 such that the scale information is displayed, for example, a second after the switch 123 is turned ON. In this case the operator can confirm the still image displayed on the monitor is adequate before the scale is displayed on the monitor. If it is not adequate, the operator can cancel storing by pushing the switch 124 or 125 before the scale information appears on the display.

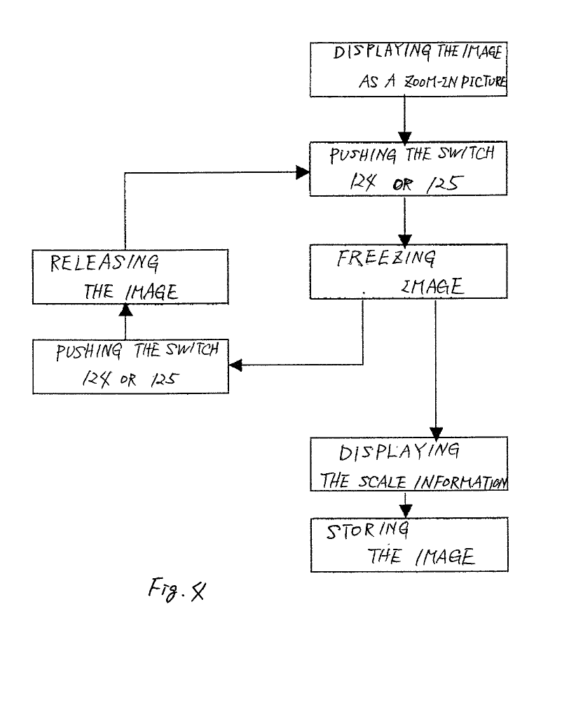

Hereinafter what is displayed in the monitor is described with reference to FIGS. 4 to FIG. 7, especially in case (4) in regard to the timing of display of scale information when the switch for storing the image is turned ON. FIG. 4 is a flowchart explaining the procedure of using the endoscope system. FIG. 5 is an illustration showing an image of an object 510 displayed as a normal picture.

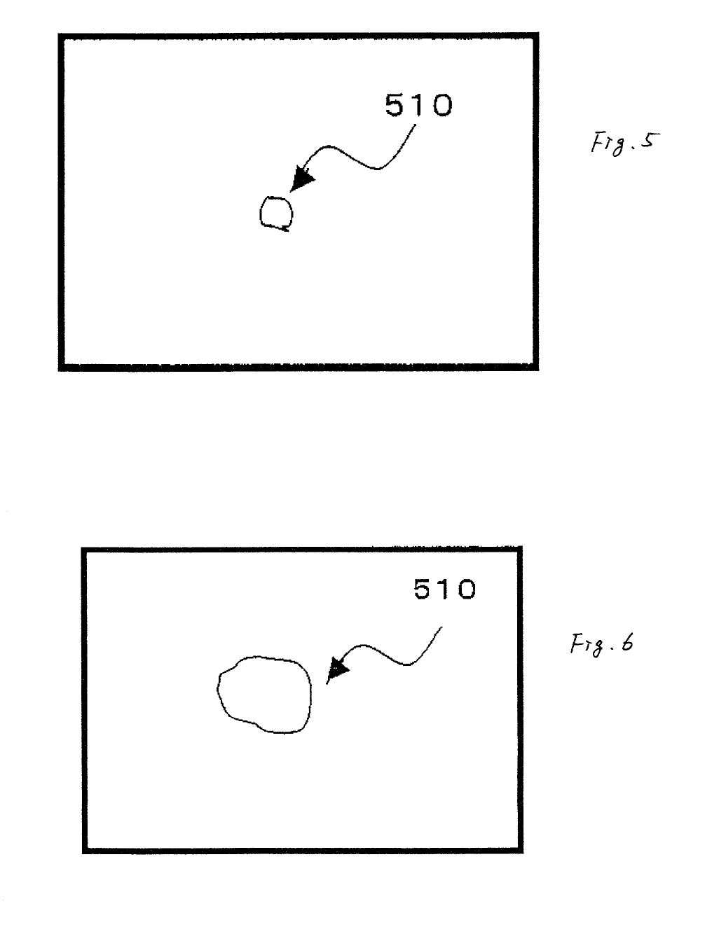

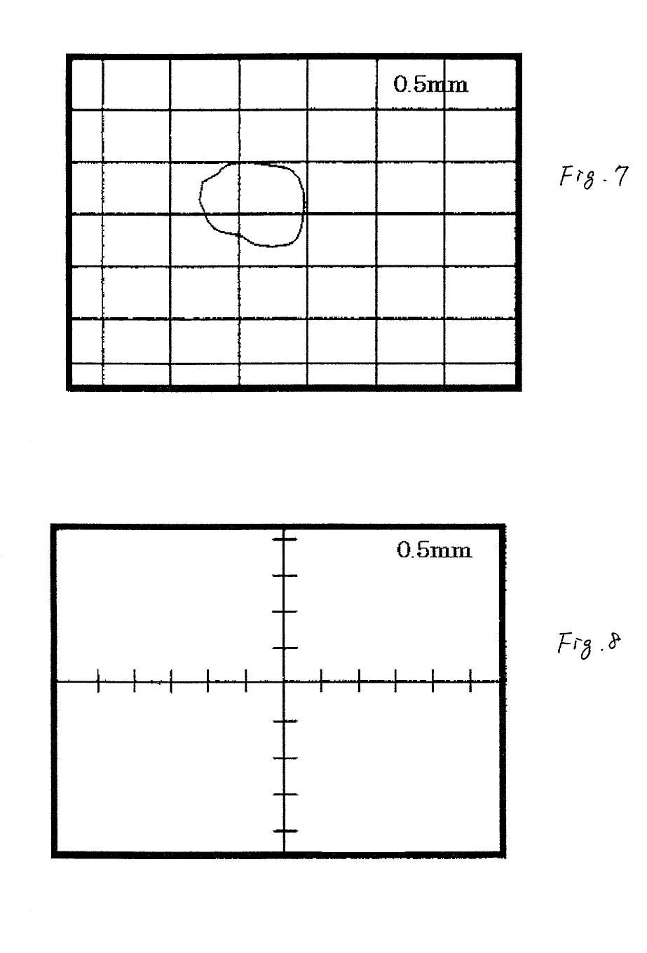

Next, the operator changes the focal length of the optical system with the lever and moves the endoscope unit 100 such that the object 510 is located within the depth of field and the image of the object 510 is displayed as a zoom-in picture, as shown in FIG. 6. Then, the operator pushes the switch 124 or 125 then the image is changed from a moving image to a still image and the still image is recorded in the camera unit 160 or disk unit 170 or printed by the printer unit 180. With a predetermined delay time after the switch 124 or 125 is turned ON, the scale information is superimposed on the image and the two are displayed together. FIG. 7 is an illustration showing the image and the scale information superimposed thereon. The scale information shown in FIG. 7 includes a number of vertically and horizontally extending lines on the monitor.

If the center of the depth of field is located at a distance from the principal points of the optical system by about 2.3 mm and the image is magnified 60 times, the preferred distance between the lines may be 30 mm on the monitor (0.5 mm for actual scale). In this case the object located at the distance of 2-2.5 mm from the principal points of the optical system can be distinguished with an error of 0.1 mm-0.2 mm.

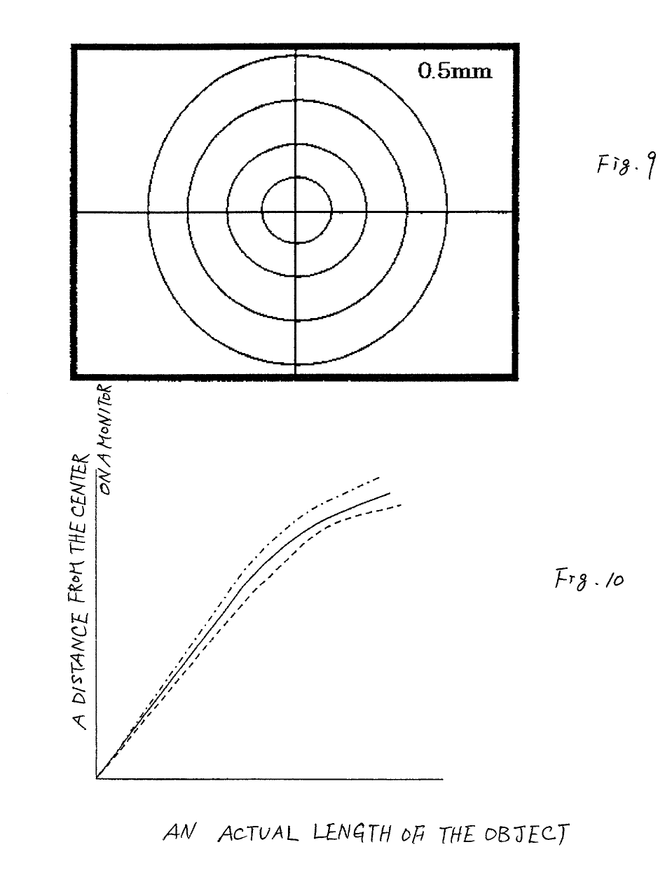

Other types of scale information may be provided as shown in FIG. 8 and FIG. 9. The scale information shown in FIG. 8 comprises two lines which are crossed orthogonally with each other and each line is graduated with a predetermined distance. The scale information shown in FIG. 9 includes coaxial circular lines, which are separated by a predetermined distance. Other types of scale information may be used too, as long as it enables measurement of the length of the object.

FIG. 10 is a graph showing a relation between a distance from the center of a monitor and actual length of the object on the condition that the object is located within the depth of field. In FIG. 10, a solid line indicates the average distortion characteristic of the optical system and dotted lines indicate the average distortion characteristic including the dispersion characteristic of the optical system. In order that the endoscope system has a wide field of view, the distortion characteristic of the optical system is designed such that the length of the object on the monitor is smaller than the actual length of the object.

FIG. 11 is another example showing scale information according to the distortion characteristic. The lines of this scale information are arranged such that a distance between the lines located at the center of the monitor is larger than the distance between the lines located at the edge of the monitor. The distance between the lines are determined according to the distortion characteristic as shown in FIG. 10. The operator can avoid misunderstanding the length of the object using this scale information.

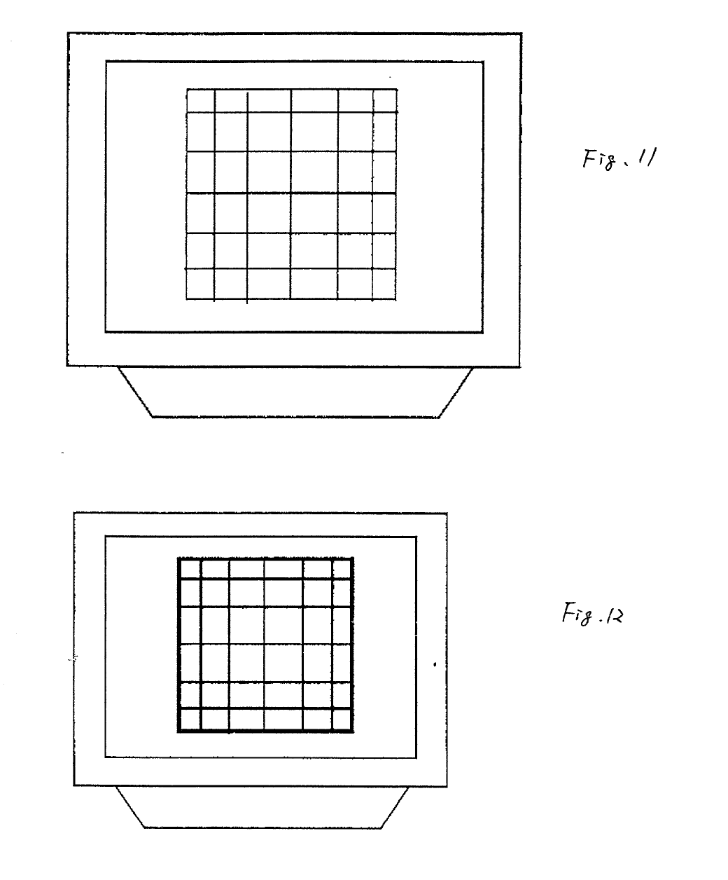

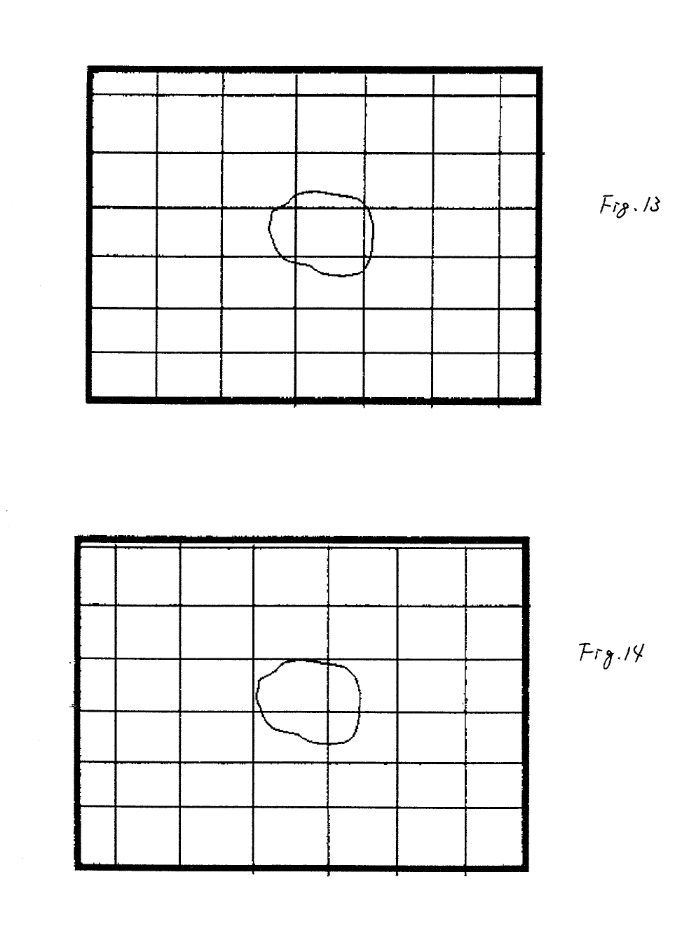

FIG. 12 is another example showing scale information according to the distortion characteristic. The width of the lines located at the center of the monitor is smaller than the width between the lines located at the edge of the monitor in this scale information. The operator would less likely to misunderstand the length of the object using this scale information.

The scale may be movable on the monitor in order that a line of the scale is located on the edge of the object.

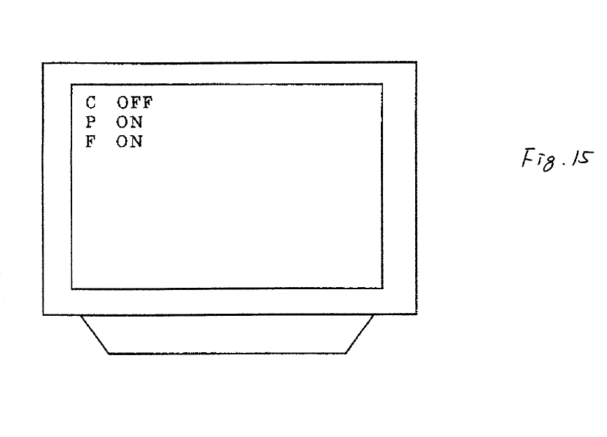

FIG. 13 is an illustration showing an image and the scale information in the case that a line of the scale information is not located on an edge of the object. In this situation it is difficult for the operator to measure the size of the object. According to the present invention, the operator can move the scale information using the directional switch 142 shown in FIG. 1 such that the line of the scale information is located on an edge of the object.

The scale information display processing unit 440 receives the signal from the directional switch 142 and the output of the scale information display processing unit 440 is fed to the superimposing circuit 430. The superimposing circuit 430 adjusts the position of the scale information according to the signal from the scale information display processing unit 440 and superimposes the scale information and the image. FIG. 14 is an illustration showing an image and the scale information that are superimposed when the scale information is moved. The superimposed image, which is the same as displayed on the monitor, is stored in the recording devices after the movement of the scale. The operator moves the scale information on the monitor, after displaying the scale information with the image. The operator then records the image with the image in the recording devices by pressing a button.

The directional switch 142 may be disposed on the operating handle. In addition, the scale information may move automatically. In this case, first the scale information moves up and down repeatedly by a distance between the adjacent lines then the operator can stop it anytime with a stop switch disposed on the operating handle (not shown). After that, the scale information moves to the right or the left automatically by a distance between the lines and then the operator stops it again. Thus, the operator can move the scale information to a desired position merely by pushing the stop switch without the use of a directional switch.

Additionally a numeric display of the distance between the adjacent lines may be provided with the scale information. In FIG. 7 and FIG. 8 the number 0.5 mm is displayed on the monitor. The operator can select whether the scale information is used or not when the image is frozen, copied and printed, respectively. If the operator prior to an examination sets that the scale information is used only when the image is frozen, the scale information is not stored when the image is copied and printed. In this case, the operator can measure the length of the object when the image is frozen.

Moreover if the operator prior to an examination sets that the scale information is used only when the image is copied or printed, the operator can observe the still image without the scale information and the scale information is copied or printed with the image. FIG. 15 is an illustration showing a menu for setting whether the scale information is used or not. In FIG. 15, C indicates copying, P is printing and F is freezing. FIG. 15 shows that the scale information is not used when the image is copied and it is used when the image is printed or frozen.

Hereinbefore the case that each switch for magnifying, freezing, coping and printing works independently is described. However the scale information may be used only when the magnifying switch and at least one of the freezing, coping or printing switches are turned ON together.

A further image processing operation indicated by the operator may trigger use of the scale information, such as enhancement processing, contrast processing or color control processing, for example.

This endoscope system can be applied to a surgical endoscope operated by an operator far from a patient. The present invention is not limited to this embodiment and can be modified if necessary.

As above described, the focal length is selectable from two levels, but the number of the levels is not limited, and more than two levels can be implemented. In this case, the scale information display processing unit 440 stores a plurality of the types of scale information, and the adequate scale information according to each focal length is used.

In the above described embodiment, each focal length has a corresponding single scale information, but a plurality of the types of scale information, for example, a distance between the lines is different such as 0.5 mm, 0.75 mm or 1 mm, can be selectively used for each focal length. The operator can select one of the scale types appropriate to the situation. Moreover the operator may select one of the scale information shown in FIG. 7 through FIG. 9.

In the above described embodiment, the varifocal lens is used in the optical system, but a zoom lens may also be used. In this case, when the operator changes magnification, the object can be observed appropriately without the moving of the endoscope unit because both the focal length and the depth of field can be adjusted independently.

According to this invention, the operator can measure the size of the object easily without detecting a distance between an endoscope part and the object.

Numerous modifications and variations of the present invention are possible in light of the above teachings. It is therefore to be understood that within the scope of the appended claims, the invention may be practiced otherwise than as specifically described herein.

What is claimed is:

1. An endoscope system, comprising:
a solid-state image pickup device having a photosensitive area and configured to produce an electronic image in response to light incident on the photosensitive area;
an optical system having a focal length and configured to transmit an optical image of an object incident onto the photosensitive area;
a superimposing device configured to superimpose on the electronic image produced by the solid-state image pickup device scale information according to the focal length of the optical system; and
a display configured to display the electronic image and the superimposed scale information.

2. The endoscope system according to claim 1, further comprising:
a mechanism configured to vary the focal length of the optical system.

3. The endoscope system according to claim 2, wherein the display is configured to display the electronic image and the scale information when the focal length is maximum.

4. The endoscope system according to claim 2, wherein the display is configured to display the electronic image and scale information in correspondence with a respective focal length.

5. The endoscope system according to claim 1, further comprising:
an image recording device configured to record the electronic image;
wherein the display displays the electronic image and the scale information when the image recording device records the electronic image.

6. The endoscope system according to claim 1, further comprising:
a processor configured to process the electronic image;
wherein the display displays the electronic image and the scale information when the processor processes the electronic image.

7. The endoscope system according to claim 6, wherein the processor includes a freeze mechanism configured to freeze the electronic image.

8. The endoscope system according to claim 1, wherein the scale information comprises at least one of plural parallel straight scale lines and plural coaxial circular scale lines.

9. The endoscope system according to claim 8, wherein a distance between the scale lines is determined according to a distortion characteristic of the optical system.

10. The endoscope system according to claim 8, wherein a width of the scale lines is determined according to a dispersion characteristic of the optical system.

11. The endoscope system according to claim 1, wherein the display displays the electronic image and the scale information in response to a predetermined operation.

12. An endoscope system having an endoscope unit and a main unit connected to the endoscope unit, comprising:
a charge coupled device disposed in the endoscope unit and having a photosensitive area configured to produce an electronic image of light incident on the photosensitive area;
an optical system disposed in the endoscope unit and including a plurality of lenses defining a focal length, configured to produce an optical image of an object incident onto the photosensitive area of the charge coupled device;
a superimposing device disposed in the main unit and electrically connected to the charge coupled device, configured to superimpose scale information according to the focal length on the electronic image produced by the charge coupled device; and
a monitor electrically connected to the superimposing device and configured to display the electronic image and the superimposed scale information.

13. The endoscope system according to claim 12, further comprising:
a wire connected to at least one of the lenses; and
a lever connected to the wire and configured to move the at least one of the lenses connected to the wire such that the focal length of the optical system is varied.

14. The endoscope system according to claim 13, wherein the monitor displays the electronic image and the scale information when the focal length is maximum.

15. The endoscope system according to claim 13, wherein the monitor is configured to display the electronic image and scale information in correspondence with a respective focal length.

16. The endoscope system according to claim 12, further comprising:
at least one of a camera unit, a disk unit and a printer unit electrically connected to the superimposing device and configured to record the electronic image;
wherein the monitor displays the electronic image and the scale information when the superimposing device stores the electronic image.

17. The endoscope system according to claim 12, further comprising:
an image processor, disposed in the main unit, for processing the electronic image;
wherein the monitor displays the electronic image and the scale information when the electronic image is processed.

18. The endoscope system according to claim 17, wherein the image processor comprises a mechanism configured produce a frozen electronic image.

19. The endoscope system according to claim 12, wherein the scale information comprises at least one of plural parallel straight scale lines and plural coaxial circular scale lines.

20. The endoscope system according to claim 19, wherein a distance between the scale lines located at a center of the monitor is smaller than a distance between the scale lines located at an edge of the monitor.

21. The endoscope system according to claim 19, wherein a width of the scale lines located at a center of the monitor is smaller than a width of the lines located at an edge of the monitor.

22. The endoscope system according to claim 12, wherein the monitor displays the electronic image and the scale information in response to a predetermined operation.

23. An endoscope system having an endoscope unit and a main unit connected to the endoscope unit, comprising:
a charge coupled device disposed at one end of the endoscope unit and having a photosensitive area configured to produce an electronic image in response to light incident on the photosensitive area;
an optical system including a plurality of lenses defining a focal length, configured to transmit an optical image of an object incident on the photosensitive area so that the charge coupled device produces a corresponding electronic image of the optical image;
a wire connected to at least one of the lenses through the endoscope unit;
a lever connected to the wire and disposed at the other end of the endoscope unit, said lever configured to move the at least one of the lenses connected to the wire such that the focal length of the optical system can be varied;
a superimposing device disposed in the main unit and electrically connected to the charge coupled device, configured to superimpose on the electronic image corresponding to the optical image scale information corresponding to the focal length; and
a monitor electrically connected to the superimposing device and configured to display the electronic image and the scale information.

24. An endoscope system having an endoscope unit and a main unit connected to the endoscope unit, comprising:
a charge coupled device disposed in the endoscope unit and having a photosensitive area configured to produce an electronic image of light incident on the photosensitive area;
means disposed in the endoscope unit and defining a focal length, for producing an optical image of an object incident onto the photosensitive area of the charge coupled device;
means disposed in the main unit and electrically connected to the charge coupled device, for superimposing scale information according to the focal length on the electronic image produced by the charge coupled device; and
a monitor electrically connected to the means for superimposing and configured to display the electronic image and the superimposed scale information.

25. The endoscope system according to claim 24, further comprising:
means for processing the electronic image;
wherein the monitor displays the electronic image and the scale information when the means for processing processes the electronic image.

26. The endoscope system according to claim 25, wherein the means for processing includes a freeze mechanism configured to freeze the electronic image.

27. The endoscope system according to claim 24, further comprising:
means for recording the electronic image.

28. The endoscope system according to claim 24, further comprising:
means for recording the electronic image and the superimposed scale information.
